(19) United States
(12) Patent Application Publication
(10) Pub. No.: US 2024/0172925 A1
(43) Pub. Date: May 30, 2024

(54) ANTI-CONTAMINATION DEVICE FOR ENDOSCOPES WITH SIDEWAYS OPTICS

(71) Applicant: ES ENDOMED SOLUTIONS GMBH, Essenbach (DE)

(72) Inventor: Jürgen KRESS, Essenbach (DE)

(21) Appl. No.: 17/754,798

(22) PCT Filed: Oct. 14, 2020
(86) PCT No.: PCT/DE2020/100889

§ 371 Date: Apr. 12, 2022

(30) Foreign Application Priority Data
Oct. 16, 2019 (DE) 10 2019 127 888.0

(51) Int. Cl. A61B 1/00

(52) U.S. Cl. CPC A61B 1/00142; A61B 1/00091; A61B 1/00179

(57) ABSTRACT

Endoscope with a contamination protection device comprising a shaft (2) which defines a longitudinal axis at the tip (21) of which a sideways-directed lens system (210), an illumination (211) and/or a jet nozzle (212) are arranged, a grip attached to a cranial end of the shaft (2) opposite the tip, a single-layer or multi-layer sheath (3) tightly enclosing at least the shaft, which is closed at a distal end (31) covering the tip of the shaft, wherein the sheath (3) is transparent at least in the area of the optics (210) and the lighting (211), wherein further the sheath seals the shaft (2) and its tip (21) at the distal end of the endoscope in a germ-proof manner.

The present invention describes a contamination protection device for an endoscope.

In gastroenterology, in addition to the classic flexible endoscopes, in which the optics and the openings for biopsy, suction and any other channels for air and water insufflation are housed at the front in the distal end of the endoscope shaft, there are also differently constructed endoscopes.

One of these variants, a so-called endoscopic retrograde cholangiopancreatography or ERCP endoscope, is used for the examination and treatment of the bile ducts. A small tube is pushed through the working channel of the endoscope and, after leaving the distal opening, further into the bile duct for the implementation of the appropriate measures. Since the bile duct ends in the duodenum, which has very cramped spatial conditions, the tip of the endoscope cannot be directed as necessary at the bile duct, which enters at an angle of approximately 90 (or more) degrees. For this reason, these devices have sideways, i.e. sideways-looking, optics at the distal end. They also have a mechanism in place there that allows the inserted tube or instrument to be angled at 90 degrees to the endoscope. This is done by a mechanically operated lever that can be raised and has a slightly curved sliding surface on which an instrument pushed through the biopsy channel in the shaft of the endoscope slides and, depending on the position of the lever, is bent more or less strongly. This lever is called an Albarran lever (named after a Spanish doctor) or elevator lever.

This mechanism, i.e. the Albarran lever, its cable with cable housing, as well as joints and excavations, is completely exposed to contact with bodily fluids during the examination. Because of the many corners and the poor accessibility when cleaning after the examination, there is a great risk of incomplete disinfection.

The present invention therefore solves the problem of infection of the endoscopes and in particular of the rugged mechanics at the distal end of the device.

An endoscope with a contamination protection device according to claim 1 is proposed as a solution. This is characterized by a germ-proof sheath or sleeve, which is placed over the equipment and has a transparent surface where the sideways optics are located. In the manner of a window, this can be a cutout in the side of a cover otherwise made of opaque material, the cutout being sealed with a transparent material, for example a plastic film, which is connected to the rest of the cover in a germ-tight manner.

In alternative embodiments, the distal end of the sheath, which lies over the distal end of the endoscope shaft and encloses it tightly, is made of a transparent material, at least in an approximately cylindrical area.

In some embodiments, the sheath can be connected in a germ-proof manner at its distal end to a cap covering the distal end of the endoscope shaft. Removable caps made of plastic to cover the mechanics are known in the prior art, but since they have openings and perforations so that they can be broken through by biopsy instruments and suction, air and/or water channels, they do not protect the distal end of the endoscope and the mechanics and optics from infection.

The present invention proposes for some embodiments that the shape of the cap, which is connected to the sheath in a germ-tight manner, resembles the known, removable plastic caps, but here the open side of the cap, i.e. the cut-out in the cap that leaves the optics and illumination source(s) free and is penetrated by an instrument emerging from the working channel of the endoscope, to be provided with a cover. This is connected to the edges of the cutout in a germ-proof manner and is therefore suitable for covering the exposed, visible mechanism, e.g. the Albarran lever, in a germ-proof manner.

This cover is in the form of a thin-walled plastic piece which completely covers the openings in the cap. It is penetrated by a thin-walled tube that fills the working channel and is connected to it in a germ-proof manner at the opening through the cover. This can already be done at the factory, i.e. the cap is delivered with the cover already connected in a germ-proof manner, or the connection is only made on site during the course of preparing the endoscope for an examination/treatment. In particular, with the latter procedure, the shaft sheath is first slipped over the endoscope shaft or unrolled like a condom, then the cap is put on, paying attention to the correct placement of the cutout above the optics and/or an instrument channel exit opening, and with an open end connected to the sheath. Subsequently or beforehand, a working channel tube lining the instrument channel, also known as the working channel, is inserted until it protrudes slightly from the mouth of the working channel in the tip of the endoscope. After these steps, the cover is placed on the open cutout of the cap, such that the working channel tube perforates the cover, and connected to both the cap and the working channel tube in a germ-tight manner.

In order to ensure an even perforation that can be easily sealed, it is proposed that the cover be provided with an opening during production, the shape and size of which is adapted to the working channel tube. In order to ensure sufficient contact area between the cover and the working channel tube in the area of the perforation, the opening is preferably provided with a circumferential collar.

Within the scope of the present invention, particularly welding or gluing are used as measures to produce a germ-proof connection. Welding is preferred for factory preparation of the contamination protection according to the invention in a ready-to-use state, whereas gluing is the simpler connection method for an on-site preparation described above, since it does not require special tools.

Underneath or within the cover, the Albarran lever can be moved. This in turn causes movement of the tube that perforates the cover and the diagnostic/therapeutic device running in it. The protection thus consists of a shaft sleeve, a distal cap, a cover for the mechanics, a tube for lining the working channel and a water/air supply tube system.

When the Albarran lever is moved/actuated, there is a mechanical load on the distal sheath of the shaft, in particular if it is designed as a cap, as proposed here. A material with sufficient elasticity must be selected for the cover so that the movement of the Albarran lever is not impaired and that the cover, the cap or the connection between the two does not tear even when the Albarran lever is deflected to its maximum extent.

A further embodiment of the contamination protection of the present invention for ERCP endoscopes includes a cap which is also designed similarly to the already known disposable caps, but the shaft tip, i.e. the entire optics and mechanics (Albarran lever and control) of the endoscope are completely covered. An additional separate cover is therefore not required. So that the optics and the lighting can also be used with such a design of the cap, this variant of the cap is provided with a highly transparent window above the endoscope optics.

This variant of the cap has a longitudinal slit in which the tube of the working channel lining can be moved lengthwise using the elevator lever thus changing its exit angle. A bellows-like and/or elastic film is located between the tube and the cap and tightly seals the cap slit. In this way, the mechanism is completely covered and protected against germs also in this embodiment. Also, the air/water channel(s) runs inside the film tube attached to the cap to cover the socket.

Under the distal caps known in the art and between the small mechanical parts there is a high potential for contamination with germs. That is why there has recently been a demand to use at least disposable one-time caps, which has already been implemented. However, this solution only solves the problem of the remaining contamination inside the cap, not that of the contamination of the mechanics. This is only advantageously made possible by the present invention.

Further advantageous developments of the invention are presented below. These can be implemented individually or in combination, provided they are not obviously mutually exclusive.

In preferred embodiments, the endoscope according to the invention with protection against contamination has a working channel for instruments and a mechanism in the shaft tip in order to influence the exit angle of an instrument led out of the working channel. In particular, this is a so-called Albarran lever with the necessary control via Bowden cables.

In further preferred embodiments, the endoscope according to the invention has one or more tubes for air and/or water insufflation. In particular, this tube can be a combined air/water tube or two separate tubes for air and water. The tubes are preferably located inside the sheath and are connected to the sheath in the area of the distal end of the sheath with an open end of the tube.

The tube preferably ends in a surface of the distal end of the sleeve that is inclined at an angle of 30-90 degrees relative to a local longitudinal direction of the shaft, particularly if this is designed as a cap. This surface is particularly preferably aligned perpendicularly to the longitudinal direction of the shaft.

The mouth of the air/water tube is shaped and directed in such a way that a water jet exiting it first hits the optics and the illumination source, or the area of the cover of the cap covering them (first embodiment of the cap) or the window of the cap covering them (second embodiment of the cap) to clean it. In addition, however, it is also preferred that the jet continues to flow as a bundled water jet even after it has touched the cap. This is achieved in particular by a flat, grazing angle of incidence of the jet on the cap above the optics.

The tube or tubes also preferably have an integrated valve at their cranial end opposite the distal end, which is shaped in such a way that it can be easily plugged into an existing valve seat of the endoscope according to the invention.

The sheath of the contamination containment of the present invention is generally single or multi-layered. In particularly preferred versions, it is in particular double-layered.

Further features, properties and advantages of the invention are apparent from the preferred exemplary embodiments presented and explained below with reference to the figures. These are for the purpose of illustration only and are not intended to limit the present invention in any way.

It is shown in

FIG. 1: In four partial figures, perspective views of the distal end of an endoscope with sideways optics, the shaft of which is covered with a protective cover of the contamination protection device according to the invention.

FIG. 2A: In four partial figures, perspective views of a cap of the contamination protection device according to the invention according to a first embodiment.

FIG. 2B: In four partial figures, perspective views of the distal end of an endoscope with sideways optics which is covered with the cap from FIG. 2A.

FIG. 2C: as FIG. 2B, but here the mechanism of the endoscope is indicated with dashed lines.

FIG. 3A: In four partial figures, perspective views of the distal end of an endoscope with the cap according to FIG. 2A, a cover and a protruding instrument channel tube.

FIG. 3B: as FIG. 3A, wherein the course of the tube inside the shaft is shown with dashed lines.

FIG. 3C: In four partial figures, further views of the distal end of the shaft with protruding instrument tube, the shaft tip inside the cap being indicated with a broken line.

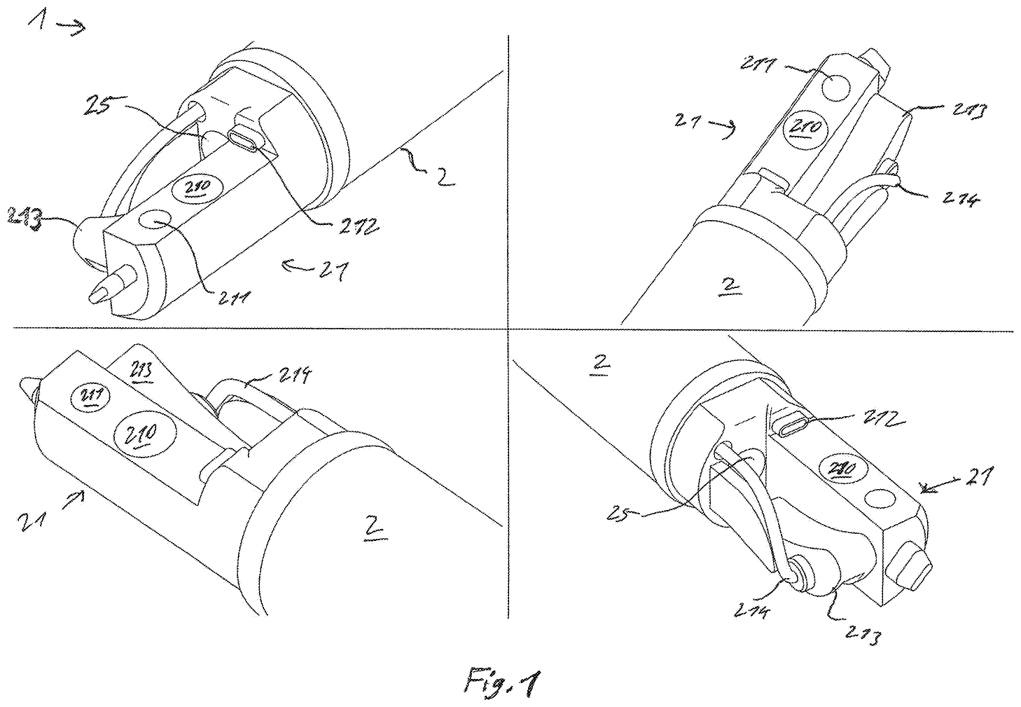

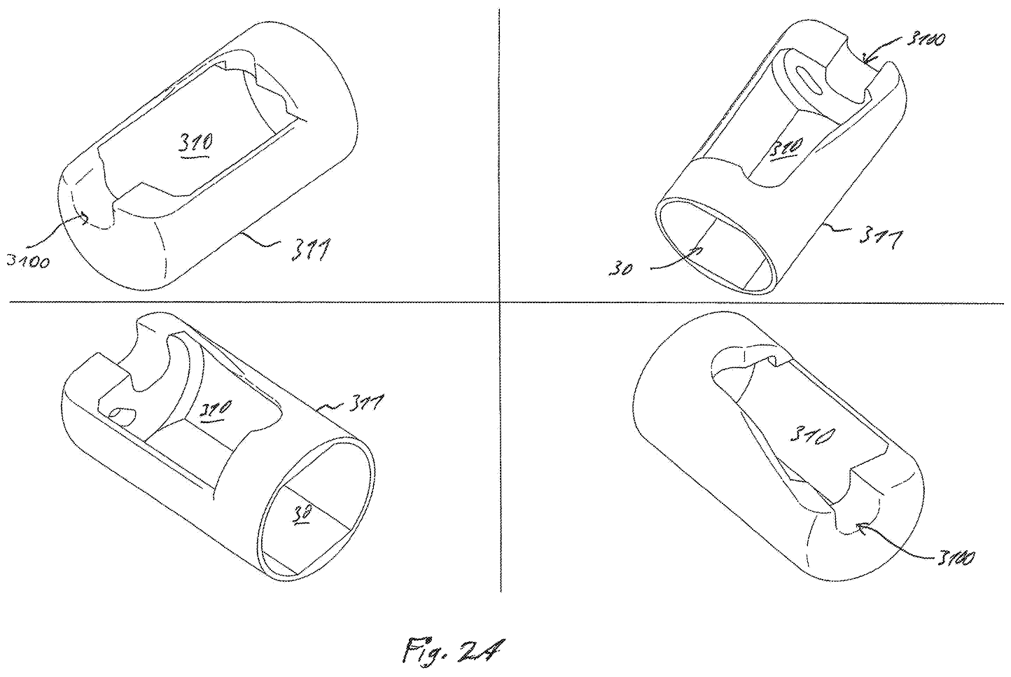

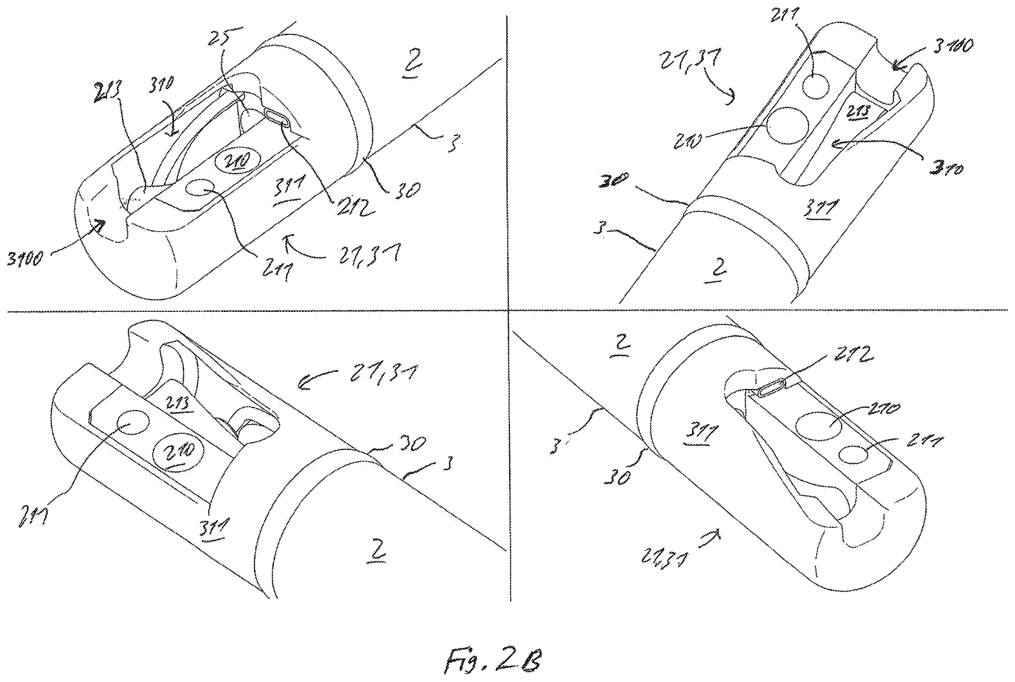

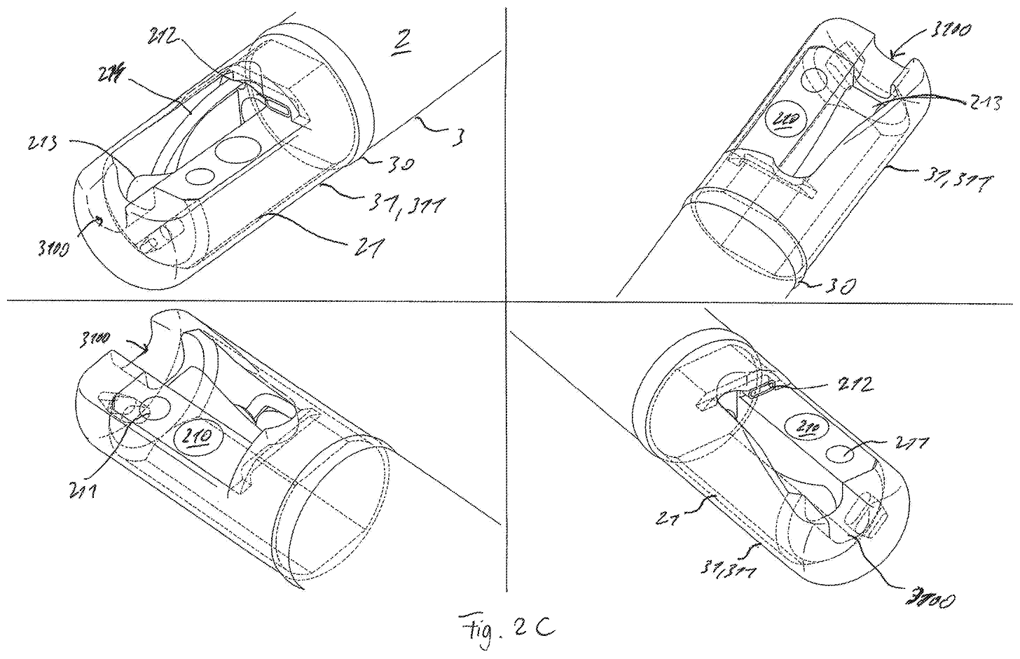

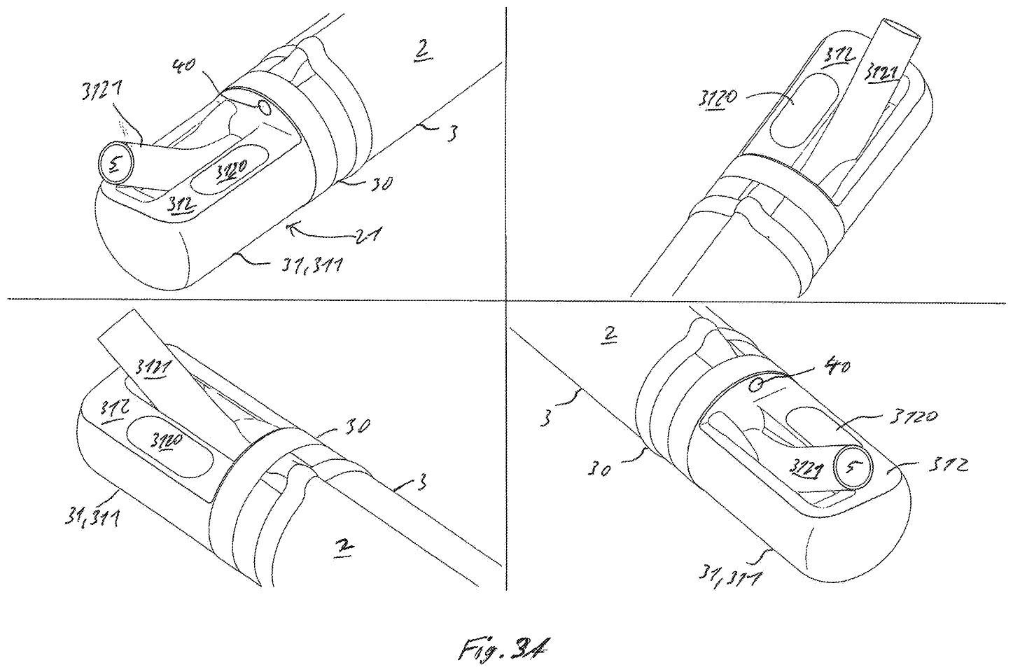

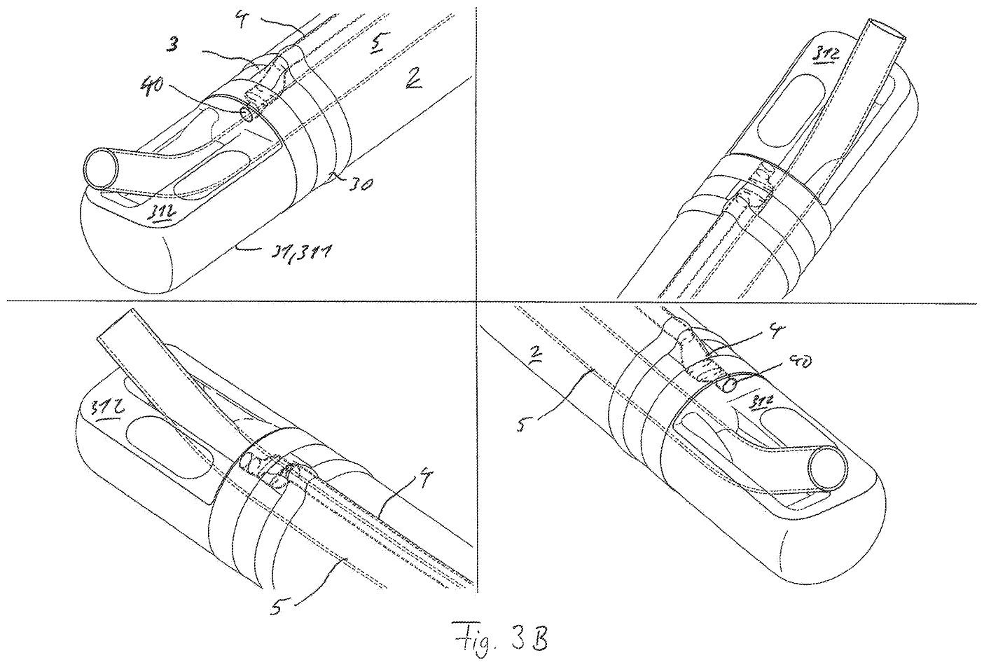

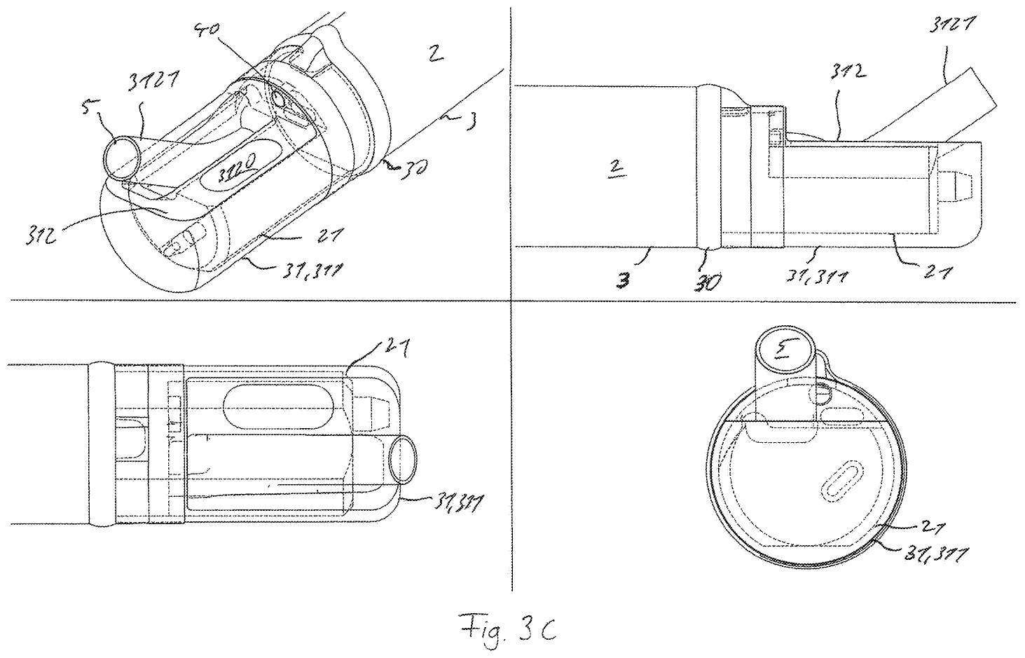

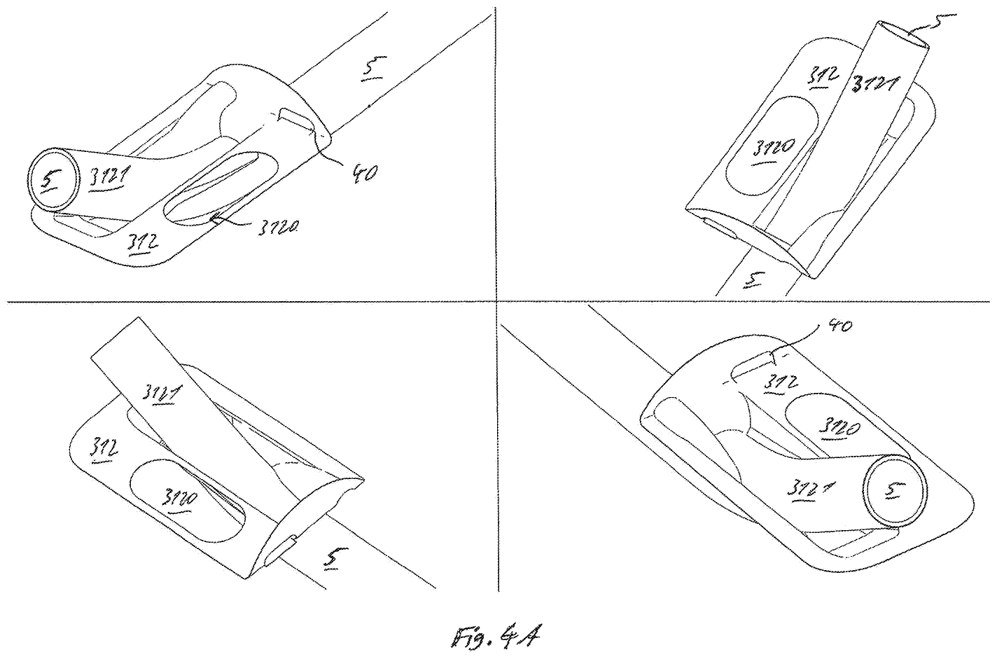

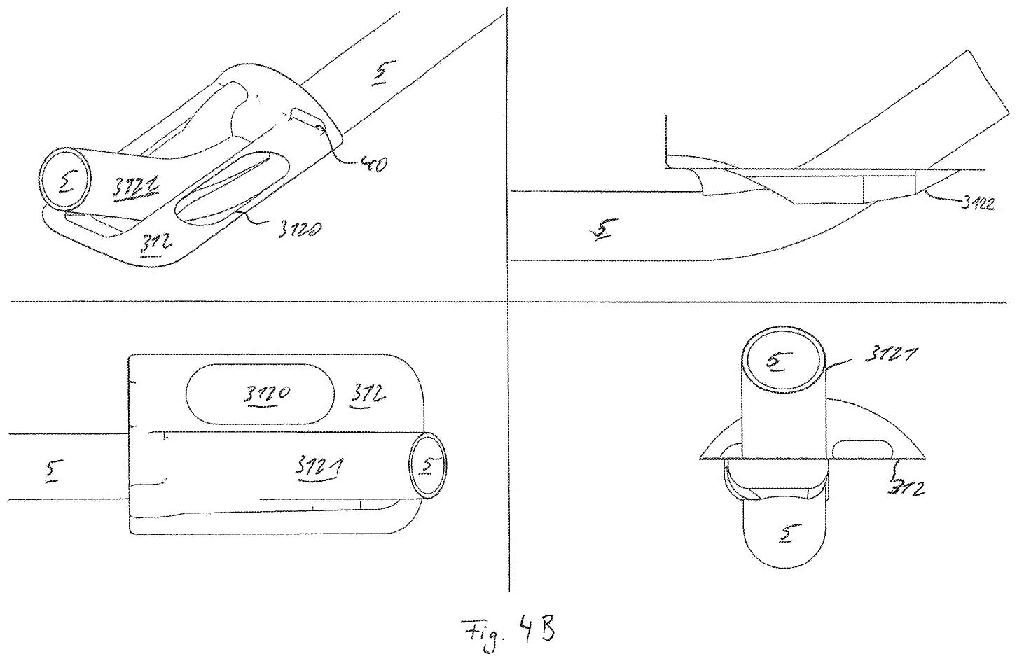

FIG. 4A: In four partial figures, perspective views of the cover of the open cutout of the first embodiment of the cap and its connection to an instrument channel tube, but without shaft or sheath.

FIG. 4B: In four partial figures, further views of the cover and its connection to an instrument channel tube.

FIG. 4C: In four partial figures, perspective views of the cover from FIGS. 4A and 4B and its connection to a suction tube.

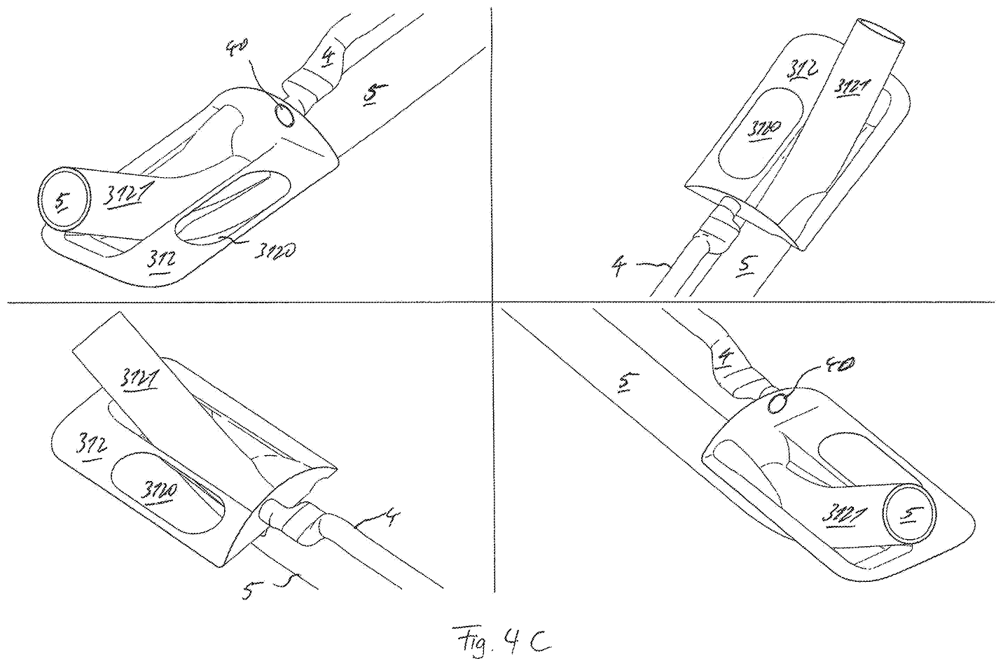

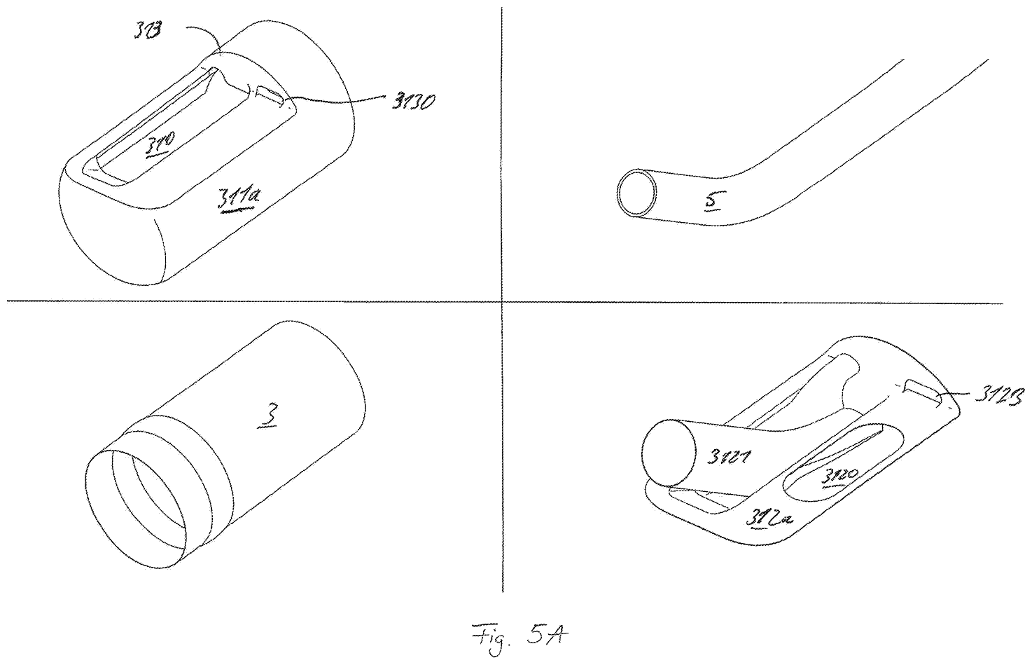

FIG. 5A: In four partial figures, perspective views of components of the contamination protection with a first variant of the cap according to the first embodiment.

FIG. 5B: In four partial figures, perspective views of components of the contamination protection with a second variant of the cap according to the first embodiment.

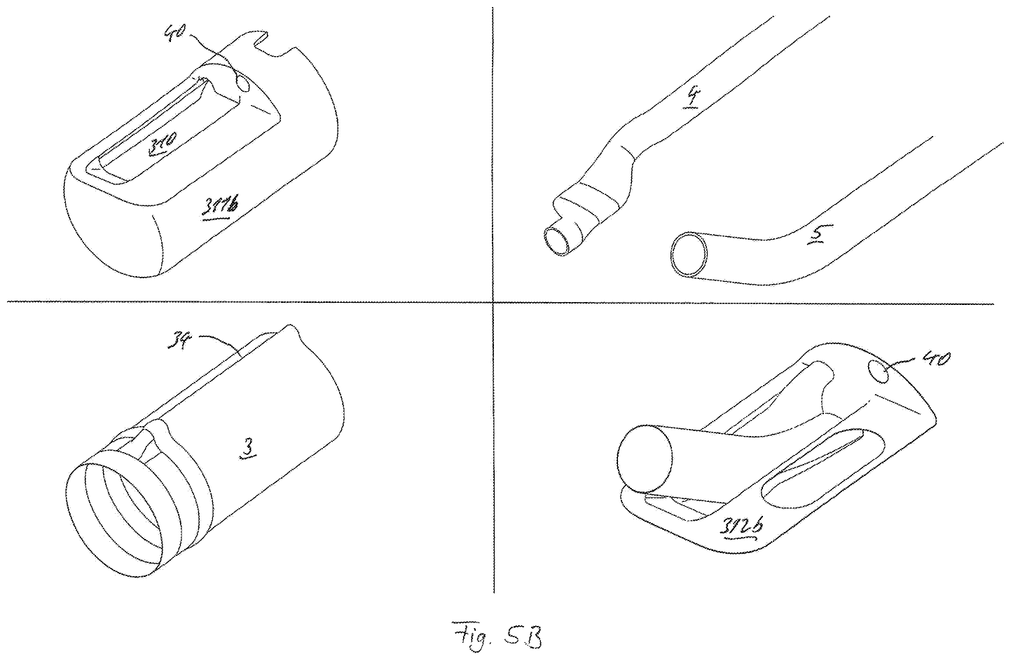

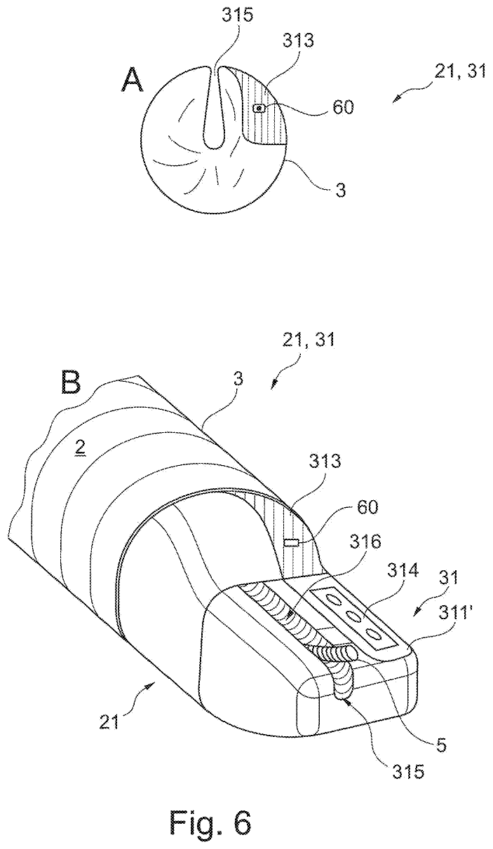

FIG. 6: In two partial figures, views of a cap of an inventive contamination protection according to a second preferred embodiment.

In FIG. 1 the shaft tip of an endoscope with sideways optics is shown in four partial figures.

Depicted are the tip 21 of the shaft 2, which here, as usual, has a round cross-section, and the sideways optics 210 in the tip 21, which is looking in a direction perpendicular to the longitudinal direction of the shaft 2, with associated, adjacent lighting or illumination source 211 which comprises a lighting means (not shown) in the form of an LED or an optical fiber through which external light is radiated. Furthermore, the mechanism can be seen, which comprises an elevator lever 213 and a Bowden cable 214 required for its movement. The opening 25 of an instrument channel and the opening 212 of an air and/or water channel inside the shaft 2 are also shown.

Due to the optics and in particular the mechanism, the shaft tip has a very jagged geometry, which makes a complete decontamination after an examination without the contamination protection according to the invention very complex, tedious and risky.

A first embodiment of the cap of a contamination protection according to the invention and its use in connection with the shaft tip is shown in FIGS. 2A-C.

FIG. 2A shows the cap 311 which has an open end 30 for plugging onto the shaft tip of an endoscope and a cutout 310 for the optics, lighting and an instrument.

In FIG. 2B, the cap 311 is shown placed on the tip of the shaft 21, being connected, in particular welded, in a germ-tight manner in the area 30 to the sheath 3, which fits closely over the shaft 2. The recess 3100 of the cutout 310 allows an instrument to be guided out of endoscope tip 21 without damaging the cap even if the elevator lever 213 only provides a minimal deflection.

In FIG. 2C, in addition to FIG. 2B, the shaft tip 21 inside the cap 311 forming the distal end 31 of the sheath 3 is also shown in dashed lines.

FIGS. 3A-C show the cap according to the first embodiment from FIG. 2A with a cover for the germ-proof closure of the open section of the cap in connection with an instrument tube of the inventive contamination protection led out of the shaft tip.

FIG. 3A shows four different perspective views of the shaft tip 21 with the cap 311 attached, forming the distal end 31 of the sheath 3. A section of the cap 311 that is not visible here is closed by the cover 312 in a completely germ-tight manner. Cover 312 is penetrated by the instrument tube 5 and is connected to it in a germ-tight manner along the contact surface of the tube-connector-like collar 3121 of the cover 312. The cover 312 has a transparent window 3120 above the optics and lighting (not visible here).

FIGS. 3B and 3C show perspective views similar to FIG. 3A, but the inner workings of the shaft 2 and the tip 21 are indicated by dashed lines. In FIG. 3B, the course of the instrument channel tube 5 in the interior of the shaft 2 can be seen, as well as the course of a suction tube 4 in the interior of the sheath 3, which covers the shaft 2 with a tight fit.

In FIG. 3C, the shaft tip 21 inside the cap 311 is indicated by a broken line.

FIGS. 4A-4C show the cover 312 with the attached instrument tube 5 and suction tube 4 (FIG. 4C) independent of the shaft tip or the cap. The cover 312 is connected to the tube in a germ-tight manner over the contact area with the collar 3121 which encloses the open end of the tube penetrating the cover 312. As shown here, it has a depression in the passage area of the tube 5, i.e. an upper side that is curved inward. This is optional and a completely smooth top with a corresponding shortening of the collar 3121 would also be possible.

FIG. 4C also shows a suction tube 4 connected to the cover in a germ-tight manner at its mouth 40.

It is proposed here that in some embodiments of the invention the cap cover and working tube and, if applicable, the suction tube as shown in FIGS. 4A-4C are provided as a pre-connected unit, so that, after putting on a cap according to the first embodiment of FIG. 2A, the instrument channel tube is inserted from the tip of the endoscope into the working or instrument channel of the shaft until the cover rests on the edges of the cutout in the cap. A germ-tight connection can then be produced there by welding or gluing in particular over the entire contact area.

FIGS. 2B-4C show the essential steps in the production of the contamination protection according to the present invention in the order of the numbers. This means that after the sheath has been pulled over the endoscope shaft (not shown), the cap is first put on (FIGS. 2A, B), connected to the open end of the sleeve in a germ-proof manner, then the instrument tube is pulled out of the working channel and the opening of the cap. (FIGS. 3A-C), and finally the cover is put on and connected in a germ-tight manner to both the cap and the working tube (FIGS. 4A, B). If a suction tube is arranged on the inside of the cover, this is connected to an opening provided as a mouth in this after the cap has been put on (FIG. 4C).

FIGS. 5A and 5B show two variants of the components of the contamination protection according to the invention when using a cap according to the first embodiment.

In both figures, the cap 311 is shown in a first sub-figure at the top left, in a second sub-figure at the bottom left is shown a section of the sheath 3 covering the endoscope shaft, in a third sub-figure at the top right are shown the tubes used and in the fourth and last sub-figure at the bottom right the germ-proof cover 312 of the open cutout 310 of the cap 311 is shown.

In the variant according to FIG. 5A, there is an elongated opening 3130 in a surface 313 oriented perpendicularly to the longitudinal direction of the shaft, which is used to connect the mouth of an air and/or water tube (not shown). Correspondingly, the cap 311a of this variant is used with a cover 312a which has a corresponding elongated opening 3123 aligned with the opening 3130 of the cap 311a.

In the variant according to FIG. 5B, the cap 311b has a round opening 40 for the germ-proof connection of a suction tube 4 instead of the elongated opening 3130 of the cap 311a for an air/water tube. The sleeve 3 has a ridge 34 for accommodating this suction tube 4, which runs along the outside of the shaft.

FIG. 6 shows in two partial figures a second embodiment of the cap which forms the distal end of the sheath of the contamination protection according to the invention. Part A shows a front view and part B shows a perspective view.

Depicted is again the distal end of the shaft 2, which in the region above the shaft tip 21 is covered by the sheath 3 in a tight-fitting manner. The distal end of the sheath 3 forms the cap 311′ with the elongated slot 315, which allows the working channel tube 5 to pass through the cap 311′, which otherwise completely covers the shaft tip 21. The free space between the tip of the shaft 21 and the edges of the slot 315 is filled with the elastic, bellows-like film 316 that follows the movements of the tube 5 and is sealed against germs.

The area 314 above the optics and the lighting is designed as a transparent window to allow the use of the optics. In the surface 313 of the distal end 31, which is oriented at a non-zero angle relative to the longitudinal direction of the shaft 2, there is the mouth 60 of an air/water insufflation, which is shaped and directed in such a way that a bundled water jet is formed, which initially cleans the window 314 above the optics and can then be used as a further focused beam for wetting an examination or operation site.

### LIST OF REFERENCE NUMERALS

1 endoscope
2 endoscope shaft
21 shaft tip
210 optics, sideways oriented
211 illumination source
212 mouth air/water channel
213 elevator lever
214 Bowden cable
3 contamination protection sheath
30 attachment area of 311 to 3
31 distal end of 3
310 cutout
3100 recess in 310
311, 311′ cap
312 cover
3120 window in 312
3121 collar
3122 depression
3123 opening for water/air insufflation
313 angled side surface with mouth
3130 mouth water/air tube in 313
314 window in 311′
315 longitudinal slit in 311′
316 bellows-like film for sealing 315
4 suction tube
40 mouth of 4, opening for 4 in 312

1. Endoscope with anti-contamination device comprising
a shaft (2), which defines a longitudinal axis and at a tip (21) of which a sideways-directed optics (210), an illumination source (211) and/or a jet nozzle (212) are arranged,
a grip attached to a cranial end of the shaft (2) opposite the tip,
a single-layer or multi-layer sheath (3) that tightly encloses at least the shaft and is closed at a distal end (31) that covers the tip of the shaft, wherein the sheath (3) is transparent at least in the area of the optics (210) and the illumination source (211),
wherein the sheath encloses the shaft (2) and its tip (21) at the distal end of the endoscope in a germ-proof manner.

2. Endoscope according to claim 1, characterized by a mechanism located in the tip (21), which comprises in particular an elevator lever (213).

3. Endoscope according to claim 1 oder 2, characterized by a tube (4) for air- and/or water insufflation, in particular a combined air/water tube or two separate tubes for air and water, which is/are
open in the vicinity of the distal end (31) of the sheath (3), and
connected there to the sheath (3) in a germ-tight manner, and
located inside the sheath (3).

4. Endoscope according to claim 3, characterized in that the tube (4), at a cranial end, is connected to an integrated valve for air/water insufflation outside the sheath, whereby the valve is fashioned to be pluggable into a valve holder of the endoscope (1).

5. Endoscope according to one of the claims 1-4 characterized in that the sheath (3) is double-layered.

6. Endoscope according to one of the claims 1-5 characterized in that the distal end (31) of the sheath (3) comprises a cap (311), which is plugged on after sliding the sheath (3) over the shaft (2) and connected to an open end (30) of the sheath (3) in a germ-tight manner, wherein the cap (31) covers the tip (21) at least on a rear side of the tip (21) opposite to an optics (210) in a germ-tight manner.

7. Endoscope according to claim 6, characterized in that the cap (311) has a cutout (310) which leaves free at least the optics (210) and the illumination source (211), which is covered by a cover (312) made of a firm, elastic, transparent and germ-proof material, which is connected to the cap (311) in a germ-tight manner along the entire circumference of the cutout (310).

8. Endoscope according to claim 7, characterized in that the cover (312) is perforated by a tube (5) placed into a working channel of the endoscope (1) and connected to the tube at the perforation point in a firm and germ-tight manner.

9. Endoscope according to claim 6, characterized in that the cap (311) completely covers the tip (21), whereby it has a transparent window (314) at least in the area of the optics (210) and the illumination source (211).

10. Endoscope according to claim 9, characterized in that the cap (311) has a slit (315) running in a longitudinal direction of the shaft (2), through which a tube (5) that is placed in a working channel of the endoscope (1) penetrates the cap (311), whereby an open region between the tube (5) and edges of the slit (315) are sealed in a germ-tight manner by means of a bellows-like and/or elastic film (316).

11. Endoscope according to one of the claims 1-10, characterized in that the air and/or water tube (4) ends in a face (313) of the cap (311) oriented at a 30 to 90 degree, in particular perpendicular, angle relative to the longitudinal axis, wherein the mouth (60) has a geometry and orientation that guides a water jet issuing from it onto the optics (210) and/or the illumination source (211).

12. Endoscope according to claim 7, characterized in that the water jet issuing from the mouth (60) remains a focused jet even beyond the optics (210) and/or the illumination source.
